(19) United States
(12) Patent Application Publication
(10) Pub. No.: US 2021/0330726 A1
(43) Pub. Date: Oct. 28, 2021

(54) Dietary herbal health supplement for the management and treatment of Parkinson's disease

(71) Applicants: Muhammad Iqbal Choudhary, Karachi (PK); Atia-tul Wahab, Karachi (PK); Humaira Zafar, Karachi (PK); Atta-ur Rahman, Karachi (PK)

(72) Inventors: Muhammad Iqbal Choudhary, Karachi (PK); Atia-tul Wahab, Karachi (PK); Humaira Zafar, Karachi (PK); Atta-ur Rahman, Karachi (PK)

(21) Appl. No.: 17/369,980

(22) Filed: Jul. 8, 2021

(51) Int. Cl. A61K 36/48; A61P 25/16; A61K 36/82; A61K 36/8962; A61K 36/52; A61K 36/87; A61K 36/61; A61K 9/00

(52) U.S. Cl. CPC A61K 36/48; A61P 25/16; A61K 36/82; A61K 9/0053; A61K 36/52; A61K 36/87; A61K 36/61; A61K 36/8962

(57) ABSTRACT

A dietary herbal health supplement to reduce the symptoms of Parkinson's disease in a human subject comprising orally administering to a subject in need thereof an effective amount of the supplement comprising Mucuna puriens L. (velvet beans), Camellia sinensis L. (green tea), Allium sativum L. (ginger), Juglan regia L. (walnut), Arachis hypogaea L. (peanut), Vitis vinifera L. (red grapes), and Eugenia caryophyllata Thunb. (clove). The supplement is safe with only natural ingredients.

### BACKGROUND OF THE INVENTION

Parkinson's disease, the most common neurodegenerative locomotion disorder, is caused by the loss of dopaminergic neurons in the substantia nigra (SN) pars compacta (SNpc). This in turn leads to accumulation of misfolded α-synuclein. Cardinal symptoms of PD include rigidity, bradykinesia, tremor, and postural abnormality, while other motor and non-motor symptoms are also associated [Dorsey et al., The Lancet Neurology, 2018, 17(11), 953; Park et al., BMC Geriatrics, 2019, 19, 1; Aghanavesi and Westin, International Conference on IoT Technologies for HealthCare, 2016, 57].

Actual cause for the onset of PD is mostly not known, however in 5 to 10% of patients, various genetic factors have been identified. Conventionally, PD is treated by dopamine replacement therapy initially, and deep brain stimulation (DBS) in later stages [Balestrino and Schapira, European Journal of Neurology, 2020, 27, 42; Blesa et al., Frontiers in Neuroanatomy, 2015, 9, 91].

The prevalence of PD in developed countries is 0.3% (in general population), while 1.0% (in people older than 60 years). The incidence of rates of PD are between 8 and 18/100,000 people, annually. Risk factors for PD includes age, gender, and some envoirmental factors including use of pesticides [Sveinbjornsdottir, Journal of Neurochemistry, 2016, 139, 324; Rizek et al., Canadian Medical Association Journal, 2016 188, 1165; Travagli et al., Nature Reviews Gastroenterology & Hepatology, 2020, 17, 685]

Symptoms of PD includes a range of motor and non-motor features, the extent of these may vary among different patients. However, the patients must reveal the fundamental clinical features and should also respond to dopaminergic therapy to fulfil the criteria for PD diagnosis. Basic motor symptoms associated with PD includes bradykinesia, tremors, rigidity, postural and gait instability, problem in speech, and blinking of eye movements [Connolly and Lang, Jama, 2014, 311, 1683; Armstrong and Okun, Jama, 2020, 323, 560].

The clinical picture among PD patients can be highly heterogeneous, leading to the subtype based on various motor symptoms. These includes postural instability and gait difficulty (PIGD), tremor dominant, or intermediate type. PD subtypes are characterized by their possible relation with prognostic aspects as well as response to treatment. For instance, tremor dominant PD show slower progression and less disability in comparison to PIGD type [Braidy et al., CNS & Neurological Disorders-Drug Targets (Formerly Current Drug Targets-CNS & Neurological Disorders, 2017, 4, 397; Solayman et al., Current Drug Metabolism, 2017, 18, 61].

In addition to motor symptoms, non-motor symptoms (NMS) are equally important to get the clear clinical picture of PD. NMS includes various conditions including problems in GI tract, sensorial, sleep, neuropsychiatric and cognitive issues, dysphagia and hyper-salivation. if non-motor symptoms are properly reported by patients and/or examined by physicians, they would have a major impact on health-related quality of life.

Currently, there is no disease modifying treatments available for PD, so symptomatic treatment is used that target dopaminergic pathway in these patients. These includes levodopa, dopamine agonist, monoamine oxidase (MAO) and catechol-O-methyl transferase (COMT) inhibitors, and anti-cholinergic drugs. Each of these therapies is associated with severe adverse effects and hence are not suitable to use for longer period. However, the disease is progressive and need longer treatment that can improve the quality of life in PD patients [Jankovic, and Aguilar, Neuropsychiatric Disease and. Treatment, 2008, 4, p.743, Rascol et al., Annals of Neurology: Official Journal of the American Neurological Association and the Child Neurology Society, 2003, 53, S15].

Therefore, there is an immense need to identify new therapeutic approaches for the treatment of PD that can modulate the pathways leading to neuronal death and dysfunction. Besides all the development in medicinal and synthetic chemistry, natural products are still considered as a major source of identifying potential candidates for various therapeutic targets. Many natural products including vegetables, fruits, and plants have been reported as efficient anti-PD agents [China et al., Biomolecules, 2019, 9, 271; Jackson et al., Frontiers in Neurology, 2019, 10, 1245]. Considering the importance of natural products for the conventional treatment of PD, we have developed a dietary herbal health supplement and analyzed its potential as anti-PD agent. There is no chemical or synthetic compound, and solvent used, except mineral water that was used blend the ingredients. This dietary herbal health supplement is potential candidate to be used in PD patients as it would safe as well as effective.

### BRIEF SUMMARY OF THE INVENTION

PD is a progressive neurodegenerative disease characterized by various motor and non-motor symptoms. The actual cause of the disease is still not explored, although various biochemical pathways and factors have been identified that lead to the on-set of disease. Currently the disease is not curable, however, it can be managed by using various anti-PD drugs including carbidopa, levodopa, anticholinergics, dopamine (DA) agonists, and monoamine oxidase type B inhibitors. The currently used conventional approaches to target Parkinson's disease are responsible to produce various adverse events in PD patients as also they have economic burden.

In view of the above stated facts, a wide literature survey was conducted, and various dietary plants reported for antioxidant, neuroprotective, anti-PD activities were selected.

This comprises of Arachis hypogaea L., Allium sativum L., Camellia sinensis L., Eugenia caryophyllata Thunb., Vitis vinifera L., Juglan regia L., and Mucuna puriens L. All the ingredients were mixed using mineral water and the supplement was prepared without the use of any organic solvent. This dietary herbal health supplement was analyzed for its potential as anti-PD agnet in in-vivo model of Parkinson's disease using male Wistar rats. The dietary herbal health supplement showed significant reduction in PD symptoms in rats. Among score 8 rats, 50% showed reduction in PD symptoms, and were decreased to score 2 and 6 (with stable postural stability, better mobility, and white furs except tremors). Among score 6 rats, all (100%) of them showed reduction in their PD symptoms, and their scores were lowered to 2. Among score 4 rats, 100% showed reduction in their PD symptoms, and were improved to score 2. Among score 2 rats, all of them (100%) showed reduction in many PD symptoms including white furs, better mobility, and stable postural ability. Only tremors were observed in these rats as they are not treatable. Acute and chronic toxicity of this dietary herbal health supplement was also conducted according to OECD guidelines at 1850, 3700, and 18500 mg/kg. The supplement was found to be safe and non-toxic as no adverse event were observed in rats after intake of the supplement.

### DETAILED DESCRIPTION OF THE INVENTION

The dietary herbal health supplement is composed of seven ingredients from dietary plant source and were combined in a specific proportion. Arachis hypogaea L., Camellia sinensis L., Vitis vinifera L., Eugenia caryophyllata Thunb. Mucuna pruriens L., and Juglan regia L. The components were mixed using mineral water. The detailed description for the selection of above-mentioned dietary plants is described below:

Literature search highlighted the anti-PD activities of garlic capsules in PD patients, hence Allium sativum L. (garlic) was included. Walnuts and peanuts have been reported for their antioxidant as well as neuroprotective activities, hence dry fruits of Juglan regia L., (Walnut) and Arachis hypogaea L. were added. Red grapes are considered as best source of antioxidants and they also help in neuronal survival, therefore, fruits of Vitis vinifera L. (red grapes) were added. Eugenia caryophyllata Thunb. (clove) is reported for its anti-PD activity, hence clove oil was used Camellia sinesis L. (green tea) is used as a good source of antioxidants as well as neuroprotective effects. It is also reported to enhance dopaminergic neuronal survival in PD rat model, hence leaves of green tea were included. Mucuna puriens L. (velvet beans) are enriched with L-DOPA and conventionally used by PD patients hence roasted beans were also added in the supplement.

### Procedure for the Preparation Of Dietary Herbal Health Supplement

The components of dietary herbal health supplement include Mucuna pruriens L. (Roasted seed powder), Camellia sinensis L. (Leaves extract), Allium sativum L. (Fresh juice of rhizomes,) Juglan regia L. (Dry fruit powder), Arachis hypogaea L. (Dry fruit powder), Vitis vinifera L. (Fresh juice), and Eugenia caryophyllata thumb. (Seed oil), all of these ingredients were mixed in a ratio of 1:1:1:0.05:1:1:1, respectively.

### Pre-Clinical Studies of Dietary Herbal Health Supplement in Rat Model of Parkinson's Disease

Humans can develop PD naturally, but it is not possible in animals. Hence, various animal models have been developed to induce PD symptoms. These include MPTP (1-methyl-4-phenyl-1,2,3,6-tetrahydropyridine), 6-OHDA (6-hydroxydopamine), and rotenone. These agents are responsible to develop nigrostriatal dopaminergic lesions in animals. In the current study, rotenone was selected as an inducer of PD symptoms in male Wistar rats [Solayman et al., Current Drug Metabolism, 2017, 1, 50; Cannon et al., Neurobiology of Disease, 2009, 34, 279]. Rotenone is a flavonoid that is found in plants, it is able to cross blood brain barrier hence also work as neurotoxic pesticide. Rotenone disrupts the biochemical pathway of electron transport chain via inhibiting complex-I in mitochondria, which ultimately lead to ROS production. It can be given via oral route, IP (intraperitoneal), or IV (intravenous).

In our study, rats were subcutaneously (s. c.) injected with 2 mg/kg rotenone continuously for 28 days. Analysis for various PD symptoms in rats was performed on daily basis including decrease mobility, piloerection, tremors and many others. Rats were categorized into different scores ranging from 2 to 10. Score 2 refer to lowest PD symptoms, while 10 refer to death due to worse PD symptoms [Wang et al., Neural Regeneration Research, 2018, 13, 112; Chen et al., Journal of Apoplexy and Nervous Diseases, 2008, 2, 17; 20; Brodell et al., BMJ Open, 2012, 2, e001971].

### Scoring Criteria for Study Design

Score 2: Mobility of rats is reduced, along with tremors and slight gait instability. Score 4: In addition to above symptoms, rats are not able to walk smoothly due to worse gait, and are rotating towards one side. Score 6: In addition to above symptoms, forelimb or hindlimb of rats become paralyzed, and are obliquely laying towards one side. Rats also show difficulty in eating and walking. Score 8: At this stage, rats become paralyzed i.e., unilateral limb (forelimb or hindlimb) paralysis, rapid weight loss, limb spasms, and inability to eat. Score 10: This is the stage, where death occur due to severity of PD symptoms.

### PD Model of Male Wistar Rats

PD model was developed in rats and pre-clinical studies were carried out after the approval of the ethical committee of ICCBS ((ASP# 2018-0007). Rats (3 months old) were randomly distributed into four groups. Control group included 10 rats and were subcutaneously injected 98% miglyol and 2% DMSO solution that served as vehicle for the experiment. Rest of the rats (217) were injected with rotenone at a dose of 2 mg/kg, consecutively for 7-28 days, on the basis of appearance of PD symptoms. All the rats were daily observed and were scored on the basis of various PD symptoms. Among them, 115 rats showed various PD symptoms, while remaining rats (102) did not show any PD symptoms and were discarded. 115 Rats with PD symptoms were further categorized into pathological group (untreated), standard group (treated with standard drug Sinemet) and supplement treated group.

### Pathological Group

Pathological group was left untreated after the appearance of PD symptoms, to analyze the rate of self-reversal of the disease symptoms such as gait instability, tremors, reduced mobility, forelimb or hindlimb paralysis) as well as non-motor symptoms such as body weight reduction, change in eating pattern, piloerection and bad furs.

Among 35 pathological control rats, 10 rats showed score 8 symptoms, 7 rats showed score 6 symptoms, 8 rats showed score 4 symptoms, and 10 rats showed score 2 symptoms.

Score 8 rats were completely paralyzed animals. Hence, the food and water were ingested by oral gavage. Among 10 rats of score 8, self-reversal was observed in two rats (20% rats), and reached to score 6, and 4. However, 8 rats (80% rats) became worsen and they reached to agonal stage (score 10 i.e, death).

Among 7 animals of score 6, minor self-reversal was observed in two rats (28.5%) and move to score 4, while one rat (14.2%) became severe and reached to score 10 (death). However, rest of four rats (57.1%) were remained at score 6.

Among eight rats of score 4, a minor self-reversal observed in four rats (50%) and reached to score 2, while four rats (50%) did not show improvement in PD symptoms and remained at score 4.

Among ten rats of score 2, all rats (100%) did not show improvement in PD symptoms i.e, tremors, piloerection, dirty fur, and gait instability and were remained at score 2. Standard drug treated group

Sinemet, a combination of Levodopa and Carbidopa, is the most common drug used for PD patients. Usually, a patient can take 1-5 tablets a day, according to the severity of the disease symptoms [20]. Hence, this drug was used as a reference drug for the current study, and the dose for the rats was selected according to the human equivalent dose.

Among 30 rats, score 8 was observed in 13 rats, score 6 was observed in 8 rats, and score 4 was observed in 9 rats.

As, rats of score 8 (13 rats) showed the severe stage of PD, this group was given highest dose i.e., 51 mg Sinemet/kg body weight. Among them, 23% rats showed improvement in PD symptoms and moved to score 6. Conversely, PD symptoms in rest of the rats (77% rats) became worsen, as they moved to score 10 (death).

Rats with score 6 were given a bit smaller dose of the drug i.e., 41 mg Sinemet/kg body weight, in comparison to score 8 animals. Half of the rats (50%) showed improvement in PD symptoms, and moved to score 4 and 2. Conversely, 25% of the rats were consistent at their PD score and did not show any betterment in their condition. While, rest of the 25% rats got worsen and were dead due to the severity of the PD symptoms.

Rats with score 4 were given 31 mg Sinemet/kg body weight, and improvement in PD symptoms was found in 22% of them. While 11% rats were found dead due to severity of PD symptoms, and rest of the 55% rats did not show any improvement and were consistent at their score.

### Dietary Herbal Health Supplement Treated Group

Among 50 rats, score 8 was observed in 16 rats, score 6 was observed in 1 rat, score 2 was observed in 6 rats, and score 2 was observed in 10 rats.

Among the supplement treated group, rats of score 8 were given the highest dose of supplement 2,000 mg/kg body weight. Among them, 50% rats showed reduction in PD symptoms and move to scores 2, and 6. Conversely, 50% rats were found dead due to severity of the PD symptoms. The results were significant, when compared with standard drug treated group as it showed reduction of PD in only 23% rats.

Score 6 rats were given a bit smaller dose of the supplement i.e., 1000 mg/kg. All the rats, 100% of the rats showed reduction in PD symptoms, and moved to score 2.

Score 4 rats were given 600 mg/kg body weight of the supplement. All the rats (100%) showed reduction in PD symptoms and moved to score 2.

Score 2 rats were also given 600 mg/kg body weight of the supplement. All the rats (100%) showed improvement in various PD symptoms such as dirty furs became white, decreased gait instability, and improved mobility. However, tremors and piloerection were still observed in these rats, therefore they were considered at score 2.

In summary, the dietary herbal health supplement was able to significantly reduce the PD symptoms in rats, in comparison to the pathological and Sinemet treated groups. This was further supported by the results of pathological group, in which 77% of the rats were found to be consistent at their respective PD scores, indicating that the reduction in PD symptoms was due to the supplement and was not a self-reversal of the disease.

### Acute and Chronic Toxicity Of Testing Dietary Herbal Health Supplement

The pre-clinical toxicological studies of dietary health supplement have been conducted to assess the safety profile as well as dose level of this novel dietary formulation to human through acute and chronic toxicity. The OECD guidelines were followed to study acute and chronic toxicity studies of dietary health supplement [Abbas et al., Bulletin of Faculty of Pharmacy, Cairo University, 2008, 56, 185].

### Protocol for the Acute and Chronic Toxicity Studies

Acute toxicity studies were conducted to evaluate the single term adverse effects of dietary herbal health supplement after oral ingestion. Animals were divided into two groups with 3 animals per sex in each group. Group I served as control, and was left untreated. Group II served as treated, and was given a single dose of 18500 mg/kg dietary health supplement, orally. Toxic signs and symptoms were carefully observed in group II rats for first 30 mins, and then after predetermined interval of 60, 120, 240, and 360 mins. Survival ratio of rats was also assessed after dosing and then at interval of 24 hours, and thereafter daily for consecutive 14 days.

Physical observation of animals included CNS and altered autonomic effects. CNS effects includes ataxia, myoclonic twitches, tremors, convulsions, and drowsiness while Altered Autonomic effect includes piloerection (Fur erection), urination, lacrimation (Red tears), salivation, and cyanosis. Their bodyweight, food consumption, and water consumption were observed during study. The death of treated animals was also observed as mortality rate.

Chronic toxicity of dietary herbal health supplement were conducted to evaluate the adverse effects occurs as a result of prolonged and continuous administration through oral gavage, consecutively for 3 months. Animals were divided into four groups with 3 animals each per sex in each group. Group I served as control, and was untreated. Group II, III, and IV animals were served as a treated, and given a doses of 18500 mg/kg dietary herbal health supplement. The physical symptoms (mentioned above) were carefully observed in group II, III, and IV animals on daily basis for the 3 months, while their body weights were recorded weekly. The rats were dissected after 3 months for testing different biochemical parameters, and gross pathology studies.

### Results of Acute Toxicity Studies

Physical parameters (mentioned above) were observed in the animals. No sign or symptoms of i.e., tremors, drowsiness or convulsions were observed. Lacrimation i.e., red tears and salivation were also not observed. Urination was normal, indicating that the dietary herbal health supplement is not inducing any adverse effect on kidneys. The food and water intake in treated rats was also found to be normal.

There were no toxicity or mortality observed within and after 14 days of dietary herbal health supplement administration. Hence, it is concluded from above observation that the dietary herbal health supplement is found to be safe at the dose of 1850, 3700, 18500 mg/kg during the acute toxicity studies.

### Results of Chronic Toxicity Studies

Three different doses of dietary herbal health supplement i.e. 1850, 3700, and 18500 mg/kg body weight (the Human Equivalent dose, double the Human Equivalent dose, and dose ten times greater than Human equivalent dose, respectively) were given orally to animals for 3 months, and observed daily for the development of any toxic signs or symptoms.

Biochemical tests, such as blood glucose, liver function test, CBC, and lipid profile were also carried out. All the biochemical parameters after the administration of dietary herbal health supplement were found to be within the normal range, indicating that the supplement was not toxic for the rats up to the dose of 3700 mg/kg. Based on the above-mentioned results it has been concluded that the dietary herbal health supplement up to the dose of 3700 mg/kg was safe to animals during the chronic toxicity studies.

Liver and kidneys were preserved in formalin for histopathological studies. The absolute and relative weights of vital organs were observed and found to be treated rats that showed no change in the relative and absolute weight of every organ in the animals, indicating that the supplement is safe to be used up to a dose of 3700 mg/kg.

What is claimed is:

1. A daily dietary supplement for management and treatment of Parkinson's disease symptoms in humans comprising one part of Mucuna puriens L. (roasted powder), one part of Camellia sinensis L. (leaf extract), one part of Allium sativum L. (fresh juice of rhizomes), one part of Juglan regia L. (dry fruit powder), one part of Arachis hypogaea L. (dry fruit powder), one part of Vitis vinifera L. (fresh juice), and 0.1 part Eugenia caryophyllata Thunb. (seed oil) in the form of tablets, capsules, suspension, and paste.

2. The supplement as in claim 1, wherein the said composition further contains suitable inactive pharmaceutical ingredients.
